(19) United States
(12) Patent Application Publication
(10) Pub. No.: US 2011/0200549 A1
(43) Pub. Date: Aug. 18, 2011

(54) Organic Compounds Suitable For Modulating Fragrance Compositions

(76) Inventors: Thierry Granier, Duebendorf (CH); Boris Schilling, Knonau (CH)

(21) Appl. No.: 13/120,941

(22) PCT Filed: Sep. 29, 2009
(86) PCT No.: PCT/CH2009/000311

§ 371 Date: Apr. 8, 2011

(30) Foreign Application Priority Data
Oct. 1, 2008 (GB) 0817933.5

(51) Int. Cl. A61L 9/01; C07C 233/05; C07C 233/01; C07C 271/10; C07D 213/56; C07D 233/64; C07C 271/18; A61K 8/33; A61Q 13/00; A24B 15/10

(52) U.S. Cl. 424/76.1; 564/218; 564/217; 560/115; 546/336; 548/338.1; 560/174; 512/26; 131/352

(57) ABSTRACT

Disclosed are compounds having the ability to modulate, namely to improve, enhance and/or modify fragrance compositions due to their ability to inhibit cytochrome P450 enzymes, e.g. CYP2A, e.g. 2A13 and 2A6, and CYP2B6.

This invention relates to a class of chemical compounds having the ability to modulate, namely to improve, enhance and or modify fragrance compositions.

The conventional way to create fragrance compositions in the fragrance industry is by the addition of chemical compounds which as such are recognised by a skilled person to possess a positive or pleasant odour themselves. In addition, chemical compounds, to be suitable as fragrances have to fulfil several criteria, for example, a low odour threshold.

Surprisingly there has been found a new class of compounds having the ability to modulate the perception of odorant compounds. Modulators are compounds that influence the olfactive perception of odorant compounds. A modulator may result in changes of intensity (overall enhancer or masking agent), quality (change of olfactive note, enhancing or masking of particular notes), duration/longevity of perception, or combinations thereof. A modulator may also enhance the overall perception of a particular odorant or mixture of odorants, or a particular olfactive quality/note.

It is believed, without being bound by theory, that the modulating effect of the compounds hereinbelow described is mainly due to the inhibition of the cytochrome P450 enzyme CYP2A13. This enzyme is predominantly expressed in the human respiratory tract, such as lung tissue, trachea and olfactory mucosa (Su et al., 2000, Cancer Res. 60: 5074-5079). It is known from the art that this enzyme is responsible for the metabolism of a number of chemical compounds, such as coumarin, a well known odorant compound, or 4-(methylnitrosamino)-1-(3-pyridyl)-1-butanone (NNK) a potent tobacco-specific nitrosamine. NNK is formed during the processing and curing of tobacco plants by nitrosation, and it is also believed that nicotine could be converted endogenously to NNK. It is present in tobacco and in tobacco smoke, both mainstream and in sidestream smoke. NNK is a procarcinogen which is metabolically activated by alpha-hydroxylation catalysed by cytochrome P450 activity and the resulting reactive electrophilic metabolites ultimately alkylate DNA.

Cytochrome P450 enzymes constitute a sub-family of heme-thiolate enzymes, which catalyse primarily mono-oxygenase reactions involving a two-stage reduction of molecular oxygen and subsequent single-oxygen atom insertion, although reductive metabolism is also known. Reactions catalysed included hydroxylation, epoxidation, N-oxidation, sulfooxidation, N-, S- and O-dealkylations, desulfation, deamination, and reduction of azo-, nitro- and N-oxide groups. In particular it has been found that most frequently hydroxylation occurs in the presence of CYP2A13, but demethylation of C-methyl and N-methyl, and epoxidation of double bonds also occur. CYP2A13 is dominantly expressed in the human nose and the respiratory tract, however, other P450 enzymes also contribute to metabolism. In particular CYP2A6 and CYP2B6 are prone to metabolize small molecular weight substrates. CYP2B6 also has been identified as being the second important catalyst besides CYP2A13 which is metabolically activating tobacco-specific nitrosamines, such as NNK (Hecht, S. S. (2008) Chem. Res. Toxicol. 21:160-171. Progress and challenges in selected areas of tobacco carcinogenesis). Examples of biochemical reactions catalysed by CYP2A13 are shown in Scheme 1.

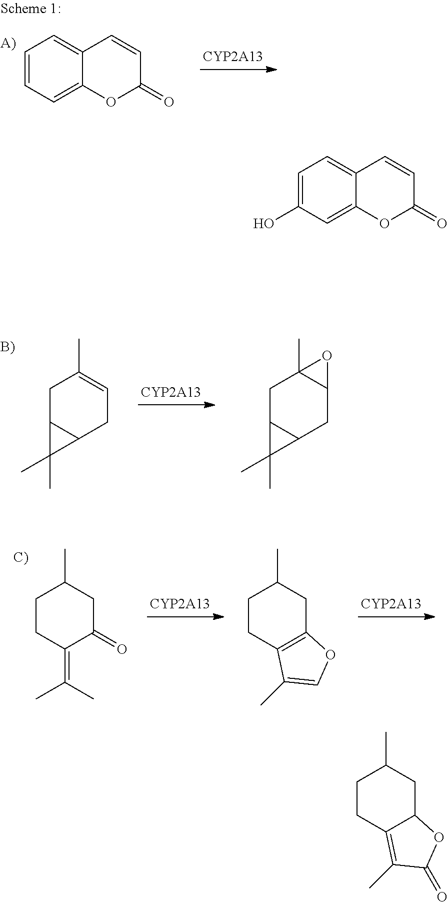

CYP2A13 is one of three members of the human CYP2A family. The other two are CYP2A6 and CYP2A7. Whereas CYP2A6 seems to be a major human liver metabolic enzyme, which also hydroxylates coumarin and metabolises nicotine to cotinine, for CYP2A7 a catalytic activity is presently unknown and it is believed to be a pseudogene.

CYP2A6 is also detected in the human respiratory tract, but CYP2A13 is the dominantly expressed isoform.

The metabolism of odorants occurring in the nose may influence olfactory sensation, and respiratory tract metabolism in general, for example in the lung tissue, may influence retronasal olfactory sensation by exchange of air passing though the respiratory tract including the nose, whereby metabolites formed by lung enzymes may reach the olfactory mucosa and receptors located therein. By inhibition of the enzymes responsible for the metabolism, in particular CYP2A13, modulation of the perception of odorant compounds in the nasal cavity can be achieved, as is shown in further detail by the examples.

Accordingly the present invention refers in one of its aspects to a compositions comprising

a) a compound of formula (I)

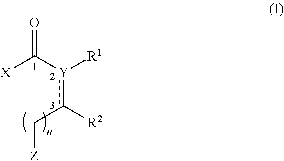

wherein n is 0 or 1;
R¹ is linear or branched C₃-C₇ alkyl, such as C₄ alkyl (n-butyl, tert. butyl, 2-methyl-(propyl), but-2-yl), C₅ alkyl (e.g. n-pentyl, 3-methyl(but-1-yl)) and C₆ alkyl (e.g. n-hexyl), benzyl or pyridylmethyl;
R² is hydrogen, C₁-C₄ alkyl (e.g. ethyl), or C₂-C₄ alkenyl (e.g. propenyl); or
R² forms together with the carbon atom to which it is attached a carbonyl group;
I) Z is a 3-6 membered monocyclic or 6-10 bicyclic hydrocarbon ring (e.g. cyclopropyl, cyclobutyl, cyclopentyl, cyclopentadienyl, cyclopentenyl, cyclohexenyl, cyclohexyl, phenyl, naphtyl) wherein up to two, i.e. 0, 1 or 2, C atom(s) are replaced by a hetero atom selected from S, O, and N (e.g. furanyl, thienyl, tetrahydrofuranyl, benzo-1,3-dioxolyl (e.g. benzo-1,3-dioxo-5-yl), pyridyl, imidazolyl);
II) Z is a 3-6 membered monocyclic or 6-10 membered bicyclic hydrocarbon ring (e.g. cyclopropyl, cyclobutyl, cyclopentyl, cyclopentadienyl, cyclopentenyl, cyclohexenyl, cyclohexyl, phenyl, naphtyl) wherein up to two, i.e. 0, 1 or 2, C atom(s) are replaced by a hetero atom selected from S, O, and N, and the ring is substituted with up to 5 groups (e.g. 1 or 2 groups) selected from hydroxyl, CN, halogen (e.g. F, Cl, Br), mono-, di-, and trihalogenomethyl (e.g. CF₃), C₁-C₃ alkoxy (e.g. methoxy, ethoxy), C₁-C₃ alkyl (e.g. ethyl), —COOR, and —OCOR wherein R is hydrogen, methyl, ethyl, propyl or isopropyl;
III) Z is a bivalent residue forming together with the C-3 a 3-6 membered monocyclic or 6-10 bicyclic hydrocarbon ring (e.g. cyclopropyl, cyclobutyl, cyclopentyl, cyclopentadienyl, cyclopentenyl, cyclohexenyl, cyclohexyl, phenyl, naphtyl) wherein up to two, i.e. 0, 1 or 2, C atom(s) are replaced by a hetero atom selected from S, O, and N (e.g. furanyl, thienyl, tetrahydrofuranyl, benzo-1,3-dioxolyl (e.g. benzo-1,3-dioxo-5-yl), pyridyl, imidazolyl);
IV) Z is a bivalent residue forming together with the C-3 a 3-6 membered monocyclic or 6-10 membered bicyclic hydrocarbon ring (e.g. cyclopropyl, cyclobutyl, cyclopentyl, cyclopentadienyl, cyclopentenyl, cyclohexenyl, cyclohexyl, phenyl, naphtyl) wherein up to two, i.e. 0, 1 or 2, C atom(s) are replaced by a hetero atom selected from S, O, and N, and the ring is substituted with up to 5 groups (e.g. 1 or 2 groups) selected from hydroxyl, CN, halogen (e.g. F, Cl, Br), mono-, di-, and trihalogenomethyl (e.g. CF₃), C₁-C₃ alkoxy (e.g. methoxy, ethoxy), C₁-C₃ alkyl (e.g. ethyl), —COOR, and —OCOR wherein R is hydrogen, methyl, ethyl, propyl or isopropyl; or
V) Z is C₁-C₄ alkoxy (e.g. ethoxy, tert-butoxy);
X is selected from hydrogen, C₁-C₃ alkyl, C₁-C₃ alkoxy, and NR³R⁴ wherein R³ and R⁴ independently are selected from hydrogen, and C₁-C₃ alkyl;
and Y represents a N- or C-atom with the proviso that
I) for X═NR³R⁴Y represents a C-atom
II) for Y═C the dotted line represents together with the carbon-carbon bond a double bond, either in E or Z configuration, or a single bond;
and
   b) at least one odorant compound.

The term “odorant compound” as used herein refers to both the volatile part of a flavour and to fragrance molecules. Examples of odorant compounds can be found e.g. in Allured's Flavor and Fragrance Materials 2004, published by Allured Publishing Inc.

Non-limiting examples are compounds of formula (I) wherein X is selected from hydrogen, methyl, and methoxy, and Y represents a N-atom.

Further non-limiting examples are compounds of formula (I) wherein Y represents a N-atom and Z is selected from cyclopropyl, phenyl, pyridyl (e.g. 2-pyridyl, 3-pyridyl and imidazol.

Further non-limiting examples are compounds of formula (I) wherein X is selected from hydrogen, methyl, and methoxy, Y represents a N-atom, and R¹ is linear C₃, C₄, C₅, C₆ or C₇ alkyl, or a branched C₃, C₄, C₅, C₆ or C₇.

Further non-limiting examples are compounds of formula (I) wherein Y represents a C-atom, X is NR³R⁴, and Z is selected from phenyl and cyclopropyl.

Further non-limiting examples are compounds of formula (I) wherein Y represents a N-atom and X is hydrogen or alkyl.

Further non-limiting examples are compounds of formula (I) wherein X is alkoxy and Y represents a N-atom.

Further non-limiting examples are compounds of formula (I) wherein R² forms together with the carbon atom to which it is attached a carbonyl group and Y represents a N-atom.

Further non-limiting examples are compounds of formula (I) wherein R² forms together with the carbon atom to which it is attached a carbonyl group, Y represents a N-atom, n=0 and Z is alkoxy.

Further non-limiting examples are compounds of formula (I) wherein X is NR³R⁴ and Y represents a C-atom.

In particular embodiments are compounds of formula (I) selected from the list consisting of N-benzyl-N-pentylacetamide, N-pentyl-N-phenylacetamide, N-butyl-N-phenylacetamide, N-pentyl-N-phenethylacetamide, N-pentyl-N-(pyridin-3-ylmethyl)acetamide, methyl pentyl(pyridin-3-ylmethyl)carbamate, N-benzyl-N-butylacetamide, methyl benzyl(butyl)carbamate, N-pentyl-N-(pyridin-4-ylmethyl)acetamide, methyl pentyl(pyridin-4-ylmethyl)carbamate, N-(cyclopropylmethyl)-N-pentylacetamide, methyl cyclopropylmethyl(pentyl)carbamate, N,N-bis(pyridin-3-ylmethyl)acetamide, methyl bis(pyridin-3-ylmethyl)carbamate, N,N-bis(pyridin-2-ylmethyl)acetamide, methyl bis(pyridin-2-ylmethyl)carbamate, N-pentyl-N-(2-(pyridin-2-yl)ethyl)acetamide, methyl pentyl(2-(pyridin-2-yl)ethyl)carbamate, N-pentyl-N-(2-(pyridin-3-yl)ethyl)acetamide, methyl pentyl(2-(pyridin-3-yl)ethyl)carbamate, N-pentyl-N-(2-(pyridin-4-yl)ethyl)acetamide, methyl pentyl(2-(pyridin-4-yl)ethyl)carbamate, N-pentyl-N-(pyridin-2-ylmethyl)acetamide, methyl pentyl(pyridin-2-ylmethyl)carbamate, N-(2-(1H-imidazol-4-yl)ethyl)-N-pentylacetamide, methyl 2-(1H-imidazol-4-yl)ethyl(pentyl)carbamate, methyl benzyl(pentyl)carbamate, N-acetyl-N-pentylcyclopropanecarboxamide, tert-butyl acetyl(pentyl)carbamate, N-benzyl-N-phenethylacetamide, N-(cyclopropylmethyl)-N-pentylformamide, (E)-2-benzylidene-N-methylheptanamide, (E)-2-benzylidene-N-methylheptanamide, (E)-2-benzylidene-N,N-dimethylheptanamide, (E)-2-(cyclopropylmethylene)-N,N-dimethylheptanamide, and (E)-2-(cyclopropylmethylene)heptanamide.

The compounds of formula (I) may comprise one or more chiral centres and as such may exist as a mixture of stereoisomers, or they may be resolved as isomerically pure forms. Resolving stereoisomers adds to the complexity of manufacture and purification of these compounds, and so it is preferred to use the compounds as mixtures of their stereoisomers simply for economic reasons. However, if it is desired to prepare individual stereoisomers, this may be achieved according to methods known in the art, e.g. preparative HPLC and GC, crystallization or by departing from chiral starting materials, e.g. starting from enantiomerically pure or enriched raw materials from the chiral pool such as terpenoids, and/or by applying stereoselective synthesis.

The compounds according to the present invention improve the performance of fragrances, or suppress or mask the perception of undesired olfactory notes of odorant compounds. By suppressing the formation of an undesired note, such as off-notes, a cleaner overall impression of the odour note can be achieved. In general, compounds of formula (I) modify the olfactive profile of a fragrance accord by altering the composition of odorant compounds that are present in the human nose, and particularly in the olfactory epithelium where they are available to olfactory receptors.

Extensive research has shown that a large number of known odorant compounds undergo a biochemical transformation in the presence of CYP2A13. Accordingly, if an CYP2A enzyme substrate is an odorant compound and the metabolite is an essentially odourless compound, a compound of less intense odor or a compound with a different odor characteristic than the odorant compound itself, then the inhibition of the enzyme will result in a slower reaction of the enzyme with the odorant compound resulting in an intensification of the overall odor or changing particular olfactive notes.

Accordingly, compounds of formula (I) are particularly well suited to be in combination with fragrance molecules that undergo a biotransformation, such as
alcohols, e.g. beta-citronellol, cedrol, Ambrinol (1,2,3,4,4a,5,6,7-Octahydro-2,5,5-trimethyl-2-naphthalenol) and nona-2,6-dienol.
aldehydes and ketones, e.g. octahydro-7-methyl-1,4-methanonaphthalen-6(2H)-one, alpha-ionone, beta-ionone, Cetone V (1-(2,6,6-trimethyl 2-cyclohexen-1-yl)-1,6-heptadien-3-one), alpha damascone, Orivone (4-(1,1-Dimethyl-propyl)-cyclohexanone) and Pulegone (5-methyl-2-(propan-2-ylidene)cyclohexanone).
ethers and acetals, e.g. methyl pamplemousse (1,1-dimethoxy-2,2,5-trimethyl-4-hexene), 1,4-cineole (1,4-epoxy-p-menthane) and rose oxyde (2-(2′-methyl-1′-propenyl)-4-methyltetrahydropyran.
esters and lactones, e.g. methyl N-methyl anthranilate, 3-phenylpropyl acetate, ethyl laiton (8-ethyl-1-oxaspiro[4.5]decan-2-one) and methyl laiton (8-methyl-1-Oxaspiro[4.5]decan-2-one).
macrocycles, e.g. Velvione® (cyclohexadec-5-ene-1-one), Habanolide® (Oxacyclohexadec-12-en-2-one) and Cosmone™ (3-methyl-5-cyclotetradecen-1-one).
heterocycles, e.g. isopropyl quinoline, pyralone (6-(1-methylpropyl)quinoline and 2-isopropyl-4-methylthiazole.
nitriles, e.g. citronellyl nitrile, cumin nitrile (4-(1-methylethyl)-benzonitrile), lemonile (3,7-dimethyl-2,6-Nonadienenitrile), terranile (3-(2,6,6-trimethyl-1-cyclohexen-1-yl)-2-propenenitrile), decanonitrile, and rose nitrile (3-(4,7,7-trimethylbicyclo[4.1.0]hept-3-ylidene)-propanenitrile).
hydrocarbons, e.g. alpha pinene, limonene, terpinolene and delta-3-carene.

Depending on the fragrance composition, there can be achieved by the addition of an effective amount of a compound of formula (I) a completely different odour note compared to a composition not comprising such a modulator. This is further illustrated by the examples.

Inhibitors, namely compounds of formula (I), can be odorants themselves and therefore can contribute to the olfactive profile of a fragrance composition in addition to inhibiting nasal- and/or respiratory tract metabolism. Such inhibitors are preferably used at concentrations at which they are not consciously perceived, namely below their sensory threshold concentration. Accordingly, compounds having a high sensory threshold are preferred; those can be used in higher concentrations without contributing themselves to the olfactive profile of a fragrance accord, while still showing modulatory effects resulting from the inhibition of P450 enzymes, in particular CYP2A13, CYP2A6, and CYP2B6.

The sensory threshold concentration is defined as the concentration of an odorant compound for which the probability of detection of the stimulus is 0.5 (that is 50% above chance, by a given individual, under the condition of the test). The sensory threshold concentration can be measured by standard methods, for example, ASTM E1432-91 and is measured either by olfactometry means or by using sniff-bottles, allowing panellists to smell the presented headspace. It is also possible to smell the presented odour in a sequential process.

Due to the fact that the compounds of formula (I) inhibit the enzyme activity of CYP2A, e.g. CYP2A6 and CYP2A13, and CYP2B6 they may also be used in combination with tobacco products to reduce or inhibit the metabolism of NNK in the respiratory tract when inhaled together with the tobacco smoke.

Accordingly, the present invention refers in a further aspect to a tobacco product, such as cigarettes, chewing tobacco, snuff tobacco, pipes tobacco and cigars, comprising at least one compound of formula (I). If used for tobacco products the addition of about 0.1 to 2% by weight, such as about 0.3 to 1% by weight, e.g. about 1% by weight based on the end product may be sufficient to achieve an effect.

Due to their properties as inhibitors for CYP2A and CYP2B enzymes, they may also be used for the regulation of nicotine metabolism in an individual, such as a nicotine replacement therapy.

Accordingly, the present invention refers in a further of its aspects to the preparation of a pharmaceutical composition comprising a compound of formula (I) as defined hereinabove.

The compounds of the present invention can be administered for, for example, oral, nasal, topical, parenteral, local or inhalant use. Oral administration includes the administration in form of tablets, capsules, chewing gums and sprays.

Furthermore, it is assumed that, if inhaled in the presence of tobacco smoke which comprise NNK, the compounds of formula (I) reduces the NNK metabolic process, because of their properties as inhibitor for CYP2A and/or CYP2B enzymes.

Accordingly, the present invention refers in a further aspect to a method comprising the step of disseminating a compound of formula (I) as defined hereinabove into a room comprising tobacco smoke. Any means capable of disseminating a volatile substance into the atmosphere may be used. The use in this specification of the term “means” includes any type of air-freshener devices which may include a heater and/or fan and nebulization systems well known to the art.

Whereas some compounds of formula (I) are known, others have never been described in literature.

Accordingly, the present invention refers in a further aspect to compounds of formula (I)

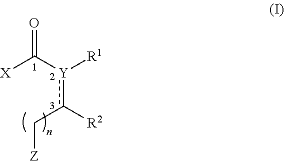

wherein n is 0 or 1;
R¹ is linear or branched C₃-C₇ alkyl, such as linear or branched C₄ alkyl (n-butyl, tert. butyl, 2-methyl-(propyl), but-2-yl), linear or branched C₅ alkyl (e.g. n-pentyl, 3-methyl(but-1-yl)) and linear or branched C₆ alkyl (e.g. n-hexyl), benzyl or pyridylmethyl;
R² is hydrogen, C₁-C₄ alkyl (e.g. ethyl), or C₂-C₄ alkenyl (e.g. propenyl); or
R² forms together with the carbon atom to which it is attached a carbonyl group;
X is selected from hydrogen, C₁-C₃ alkyl (e.g. ethyl), C₁-C₃ alkoxy, and NR³R⁴ wherein R³ and R⁴ independently are selected from hydrogen, and C₁-C₃ alkyl; with the proviso that if R¹ is pyridylmethyl X is not methyl;
I) Z is a 3-6 membered monocyclic or 6-10 bicyclic hydrocarbon ring (e.g. cyclopropyl, cyclobutyl, cyclopentyl, cyclopentadienyl, cyclopentenyl, cyclohexenyl, cyclohexyl, phenyl, naphtyl) wherein one or two C atom(s) are replaced by a hetero atom selected from S, O, and N (e.g. furanyl, thienyl, tetrahydrofuranyl, benzo-1,3-dioxolyl (e.g. benzo-1,3-dioxo-5-yl), pyridyl, imidazolyl); with the proviso that for Z=pyridyl R¹ is not benzyl;
II) Z is a 3-6 membered monocyclic or 6-10 membered bicyclic hydrocarbon ring (e.g. cyclopropyl, cyclobutyl, cyclopentyl, cyclopentadienyl, cyclopentenyl, cyclohexenyl, cyclohexyl, phenyl, naphtyl) wherein one or two C atom(s) are replaced by a hetero atom selected from S, O, and N, and the ring is substituted with up to 5 groups (e.g. 1 or 2 groups) selected from hydroxyl, CN, halogen (e.g. F, Cl, Br), mono-, di-, and trihalogenomethyl (e.g. CF₃), C₁-C₃ alkoxy (e.g. methoxy, ethoxy), C₁-C₃ alkyl (e.g. ethyl), —COOR, and —OCOR wherein R is hydrogen, methyl, ethyl, propyl or isopropyl;
III) Z is C₁-C₄ alkoxy (e.g. ethoxy, tert-butoxy); with the proviso that for Z=ethoxy R¹ is not benzyl;
or
IV) Z is cyclopropyl;
and Y represents a N- or C-atom with the proviso that for X is NR³R⁴, Y═C and the dotted line represents together with the carbon-carbon bond a double bond, either in E or Z configuration, or a single bond.

Amides, i.e. the compounds of formula (I) wherein Y represents a N-atom and X is hydrogen or alkyl, may be prepared either by acylation (e.g. by acetylation, or formylation following the general procedure known to the person skilled in the art) of the appropriate secondary amine or by N-alkylation of the appropriate N-monosubstituted amide.

Carbamates, i.e. the compounds of formula (I) wherein X is alkoxy and Y represents a N-atom, may be obtained by acylation of the appropriate secondary amine (e.g. in the presence of ClCO₂Me).

Imides, i.e. the compounds of formula (I) wherein R² forms together with the carbon atom to which it is attached a carbonyl group and Y represents a N-atom, and N-acylcarbamates, i.e. the compounds of formula (I) wherein R² forms together with the carbon atom to which it is attached a carbonyl group, Y represents a N-atom, n=0 and Z is alkoxy, may be prepared by acylation of the appropriate N-monosubstituted amide.

α,β-Unsaturated amides, i.e. compounds of formula (I) wherein X is NR³R⁴ and Y represents a C-atom, may be prepared by aminolysis of the appropriate α,β-unsaturated ester.

The invention is now further described with reference to the following non-limiting examples. These examples are for the purpose of illustration only and it is understood that variations and modifications can be made by one skilled in the art.

### EXAMPLE 1

### N-benzyl-N-pentylacetamide

A mixture of benzylamine (15 g, 0.14 mol) and n-pentyliodide (7.1 g, 0.035 mol) was refluxed for 1 h, cooled, poured into water, and extracted three times with diethyl ether (70 ml). The combined organic phases were washed three times with a saturated aqueous NaCl solution, dried (MgSO₄), and the solvent evaporated. Ball-to-ball distillation (70° C., 0.1 mbar) of the crude product (12 g) gave N-benzyl-N-pentylamine (4.8 g) that was dissolved in CH₂Cl₂ (30 ml), cooled to 0° C., and treated with Et₃N (4.3 ml, 31.2 mmol) and with a solution of acetyl chloride (2.29 g, 28.6 mmol) in CH₂Cl₂ (20 ml). The resulting solution was stirred at 20° C. for 2 h, cooled to 0° C., poured into a saturated aqueous solution of NaHCO₃ and ice, and extracted three times with hexane (90 ml). The combined organic phases were washed twice with a saturated aqueous NaCl solution, dried (MgSO₄), and the solvent evaporated. FC (Flash Chromatography) (300 g SiO₂, hexane/methyl t-butyl ether 1:1) of the crude product (5.8 g) gave N-benzyl-N-pentylacetamide (3.4 g, 43%). Boiling point: 137° C. (0.08 mbar).

¹³C-NMR (100 MHz, CDCl₃): δ (two rotamers) 170.80 and 170.45 (2s, 1C), 137.84 and 137.03 (2s, 1C), 128.84 (d), 128.47 (d), 127.97 (d), 127.46 and 127.18 (2d, 1C), 126.17 (d), 51.90 and 48.02 (2t, 1C), 47.88 and 46.04 (2t, 1C), 29.11 and 28.93 (2t, 1 C), 28.03 and 27.20 (2t, 1C), 22.43 and 22.32 (2t, 1C), 21.79 and 21.40 (2q, 1C), 13.96 and 13.90 (2q, 1C).

MS (EI): 219 (17), 204 (10), 190 (1), 176 (2), 162 (6), 148 (7), 128 (6), 120 (65), 106 (37), 91 (100), 86 (7), 65 (12), 43 (21).

### EXAMPLE 2

### N-pentyl-N-phenylacetamide

A mixture of acetanilide (6 g, 44.4 mmol) in DMSO (30 ml) was treated with powdered KOH (3 g, 53.3 mmol). The resulting mixture was cooled to 0° C., treated with n-pentyliodide (10.85 g, 53.3 mmol), stirred at 0° C. for 2 h and at 20° C. for 2.5 h, cooled to 0° C., poured into ice-water (100 ml), and extracted three times with hexane (100 ml). The combined organic phases were washed three times with a saturated aqueous NaCl solution, dried (MgSO₄) and the solvent evaporated. Ball-to-ball distillation (0.08 mbar) of the crude product (9.8 g) followed by FC (280 g SiO₂, hexane/methyl t-butyl ether 2:1) of the fraction distilling at 100-110° C. (7.2 g) gave N-pentyl-N-phenylacetamide (4.97 g, 55%). Boiling point: 100° C. (0.08 mbar).

¹³C-NMR (100 MHz, CDCl₃): δ 170.05 (s), 143.23 (s), 129.58 (d, 2C), 128.11 (d, 2C), 127.71 (d), 48.98 (t), 28.92 (t), 27.41 (t), 22.80 (q), 22.37 (t), 13.94 (q).

MS (EI): 205 (7), 204 (1), 190 (1), 163 (1), 162 (2), 135 (19), 120 (5), 106 (100), 93 (14), 77 (13), 43 (14).

### EXAMPLE 3

### N-pentyl-N-phenethylacetamide

A mixture of N-acetyl-2-phenylethylamine (3 g, 18 mmol), NaH (0.9 g, 20 mmol), and n-pentyliodide (7.3 g, 37 mmol) in THF (30 ml) was refluxed for 1 h, cooled to 0° C., poured into ice-cold 2M aq. HCl (50 ml), and extracted twice with MTBE (50 ml). The combined organic phases were washed with water (50 ml), with a saturated aqueous NaCl solution (50 ml), dried (MgSO₄) and the solvent evaporated. FC (90 g SiO₂, hexane/methyl t-butyl ether 1:1) of the crude product (7.8 g) gave N-pentyl-N-phenethylacetamide (2.03 g, 47%). Boiling point: 150° C. (0.07 mbar).

¹³C-NMR (100 MHz, CDCl₃): δ (55:45 rotameric mixture) 170.20 (s), 170.16 (s), 139.42 (s), 138.25 (s), 128.80 (d, 2C), 128.74 (d, 2C), 128.70 (d, 2C), 128.42 (d, 2C), 126.71 (d), 126.22 (d), 50.34 (t), 49.48 (t), 48.00 (t), 45.62 (t), 35.29 (t), 34.07 (t), 29.17 (t), 28.92 (t), 28.59 (t), 27.39 (t), 22.48 (t), 22.38 (t), 21.55 (q), 21.31 (q), 14.00 (q), 13.93 (q).

MS (EI): 233 (6), 190 (1), 176 (1), 142 (37), 134 (5), 105 (12), 100 (100), 91 (9), 44 (26), 43 (27).

### EXAMPLE 4

### N-pentyl-N-(pyridin-3-ylmethyl)acetamide

Prepared as described in Example 1 from 3-(aminomethyl)pyridine and n-pentyliodide via N-(pyridin-3-ylmethyl)pentan-1-amine (58%, after ball-to-ball distillation at 120° C., 0.06 mbar of the crude product). Acetylation gave after FC (SiO₂, methyl t-butyl ether/methanol 10:1) N-pentyl-N-(pyridin-3-ylmethyl)acetamide (54%). Boiling point: 184° C. (0.06 mbar).

¹³C-NMR (100 MHz, CDCl₃): δ (main rotamer, 74%) 170.66 (s), 149.05 (d), 148.56 (d), 136.06 (d), 133.86 (s), 123.60 (d), 48.31 (t), 45.94 (t), 28.85 (t), 28.20 (t), 22.30 (t), 21.33 (q), 13.88 (q). δ (minor rotamer, 26%) 170.51 (s), 149.11 (d), 148.25 (d), 133.65 (d), 132.60 (s), 123.69 (d), 49.62 (t), 46.02 (t), 29.03 (t), 27.16 (t), 22.38 (t), 21.80 (q), 13.93 (q).

MS (EI): 220 (12), 205 (16), 191 (3), 177 (5), 163 (6), 149 (6), 135 (11), 128 (4), 121 (100), 107 (33), 93 (31), 92 (50), 65 (19), 43 (27).

### EXAMPLE 5

### methyl pentyl(pyridin-3-ylmethyl)carbamate

Prepared as described in Example 4 from 3-(aminomethyl)pyridine and n-pentyliodide via N-(pyridin-3-ylmethyl)pentan-1-amine. Acylation using methyl chloroformate gave after FC (SiO₂, methyl t-butyl ether/hexane 1:1) methyl pentyl(pyridin-3-ylmethyl)carbamate (75%). Boiling point: 182° C. (0.06 mbar).

¹³C-NMR (100 MHz, CDCl₃): δ (main rotamer) 157.40 (br. s), 148.95 (br. d), 148.67 (d), 135.79 (br. d), 133.72 (s), 123.53 (d), 52.74 (q), 48.11 (br. t), 46.61 (br. t), 28.85 (t), 27.81 (br. t), 22.32 (t), 13.92 (q).

MS (EI): 236 (10), 221 (1), 205 (1), 180 (7), 179 (66), 107 (6), 93 (11), 92 (100), 65 (16), 59 (5).

### EXAMPLE 6

### N-benzyl-N-butylacetamide

Prepared as described in Example 1 from benzylamine and n-butyliodide via N-benzyl-N-butylamine (50%, after ball-to-ball distillation at 100° C., 0.08 mbar of the crude product). Acetylation gave after FC (SiO₂, methyl t-butyl ether/hexane 1:1) N-benzyl-N-butylacetamide (73%). Boiling point: 126° C. (0.06 mbar).

¹³C-NMR (100 MHz, CDCl₃): δ (55:45 rotameric mixture) 170.83 and 170.45 (2s, 1C), 137.83 and 137.03 (2s, 1C), 128.85 (d), 128.47 (d), 127.96 (d), 127.46 and 127.19 (2d, 1C), 126.17 (d), 51.89 and 48.02 (2t, 1C), 47.64 and 45.81 (2t, 1C), 30.46 and 29.64 (2t, 1C), 21.80 and 21.39 (2q, 1C), 20.18 and 20.01 (2t, 1C), 13.83 and 13.73 (2q, 1 C).

MS (EI): 205 (19), 190 (10), 162 (5), 148 (5), 120 (57), 106 (38), 91 (100), 72 (12), 65 (14), 43 (21).

### EXAMPLE 7

### methyl benzyl(butyl)carbamate

Prepared as described in Example 6 from benzylamine and n-butyliodide via N-benzyl-N-butylamine. Acylation using methyl chloroformate gave after FC (SiO₂, methyl t-butyl ether/hexane 3:7 to 1:1) methyl benzyl(butyl)carbamate (38%). Boiling point: 115° C. (0.13 mbar).

¹³C-NMR (100 MHz, CDCl₃): δ (rotameric mixture) 157.40 (br. s), 138.04 (s), 128.48 (d, 2 C), 127.79 (br. d), 127.20 (d, 2C), 52.57 (q), 50.32 and 49.98 (2t, 1C), 46.77 and 45.74 (2t, 1C), 30.16 and 29.80 (2t, 1C), 19.95 (t), 13.77 (q).

MS (EI): 221 (10), 206 (1), 189 (1), 179 (4), 178 (33), 92 (8), 91 (100), 65 (8), 59 (3), 42 (2), 41 (3).

### EXAMPLE 8

### N-pentyl-N-(pyridin-4-ylmethyl)acetamide

Prepared as described in Example 1 from 4-(aminomethyl)pyridine and n-pentyliodide via N-(pyridin-4-ylmethyl)pentan-1-amine (27%, after ball-to-ball distillation at 120-140° C., 0.08 mbar of the crude product). Acetylation gave after FC (SiO₂, methyl t-butyl ether/EtOAc 9:1) N-pentyl-N-(pyridin-4-ylmethyl)acetamide (20%). Boiling point: 150° C. (0.09 mbar).

¹³C-NMR (100 MHz, CDCl₃): δ (main rotamer, 70%) 170.73 (s), 149.93 (d, 2C), 146.94 (s), 122.50 (d, 2C), 48.78 (t), 47.70 (t), 28.86 (t), 28.24 (t), 22.28 (t), 21.23 (q), 13.85 (q). δ (minor rotamer, 30%) 170.75 (s), 150.33 (d, 2C), 146.44 (s), 121.12 (d, 2C), 51.08 (t), 46.48 (t), 29.03 (t), 27.26 (t), 22.38 (t), 21.67 (q), 13.91 (q).

MS (EI): 220 (7), 205 (17), 191 (2), 177 (2), 163 (5), 149 (3), 135 (3), 128 (4), 121 (100), 107 (13), 93 (21), 92 (25), 65 (11), 43 (24).

### EXAMPLE 9

### methyl pentyl(pyridin-4-ylmethyl)carbamate

Prepared as described in Example 8 from 4-(aminomethyl)pyridine and n-pentyliodide via N-(pyridin-4-ylmethyl)pentan-1-amine. Acylation using methyl chloroformate gave after FC (SiO₂, methyl t-butyl ether/hexane 2:1) methyl pentyl(pyridin-4-ylmethyl)carbamate (81%). Boiling point: 140° C. (0.09 mbar).

¹³C-NMR (100 MHz, CDCl₃): δ 157.41, 156.66 (br. s, 1C), 149.79 (d, 2C), 147.49 (s), 122.37 (br. d), 122.37 (br. d), 52.78 (br. q), 49.72, 49.44 (br. t, 1C), 47.81, 47.05 (br. t, 1C), 28.80 (t), 27.64 (br. t), 22.30 (t), 13.89 (q).

MS (EI): 236 (12), 221 (1), 205 (1), 180 (11), 179 (100), 147 (5), 107 (29), 106 (14), 93 (10), 92 (48), 65 (18), 59 (13).

### EXAMPLE 10

### N-(cyclopropylmethyl)-N-pentylacetamide

A mixture of N-(n-pentyl)acetamide (0.6 g, 4.7 mmol), and NaH (0.23 g, 5.2 mmol) in DME (10 ml) was treated dropwise with a solution of bromomethylcyclopropane (1 g, 7.1 mmol) in DME (5 ml) and the resulting mixture was heated at 60° C. for 19 h, cooled to 0° C., poured into ice-cold 2M aq. HCl (20 ml), and extracted twice with MTBE (30 ml). The combined organic phases were washed with water (30 ml), with a saturated aqueous NaCl solution (30 ml), dried (MgSO₄) and the solvent evaporated. FC (100 g SiO₂, hexane/methyl t-butyl ether 1:1) of the crude product (1.1 g) gave N-(cyclopropylmethyl)-N-pentylacetamide (0.76 g, 84%). Boiling point: 120° C. (0.11 mbar).

¹³C-NMR (100 MHz, CDCl₃): δ (main rotamer, 70%) 170.21 (s), 49.41 (t), 48.64 (t), 29.02 (t), 28.49 (t), 22.37 (t), 21.43 (q), 13.93 (q), 9.76 (d), 3.60 (t, 2C). δ (minor rotamer, 30%) 169.89 (s), 53.00 (t), 45.86 (t), 29.20 (t), 27.24 (t), 22.45 (t), 21.73 (q), 13.97 (q), 10.43 (d), 3.72 (t, 2C).

MS (EI): 183 (2), 168 (9), 154 (55), 140 (16), 127 (16), 126 (34), 112 (11), 98 (20), 84 (100), 70 (13), 56 (30), 55 (64), 43 (55).

### EXAMPLE 11

### methyl cyclopropylmethyl(pentyl)carbamate

A mixture of methyl pentylcarbamate (2.0 g, 14 mmol, prepared from pentylamine and methyl chloroformate), and NaH (55%, 0.6 g, 28 mmol) in DME (40 ml) was treated dropwise with a solution of bromomethylcyclopropane (2.7 g, 19 mmol) in DME (10 ml) and the resulting mixture was heated at 60° C. for 5 h, treated with bromomethylcyclopropane (0.9 g, 7 mmol), heated at 60° C. for 2 h, cooled to 0° C., poured into ice-cold 2M aq. HCl (20 ml), and extracted twice with MTBE (70 ml). The combined organic phases were washed with water (70 ml), with a saturated aqueous NaCl solution (70 ml), dried (MgSO₄) and the solvent evaporated. FC (90 g SiO₂, hexane/methyl t-butyl ether 5:1) of the crude product (2.82 g) gave methyl cyclopropylmethyl(pentyl)carbamate (1.8 g, 66%). Boiling point: 150° C. (0.09 mbar).

¹³C-NMR (100 MHz, CDCl₃): δ 156.82 (s), 52.14 (q), 51.38 (br. t), 47.09 (br. t), 28.94 (t), 27.89 (br. t), 22.36 (t), 13.89 (q), 10.11 (br. d), 3.38 (t, 2C).

MS (EI): 199 (2), 184 (6), 170 (4), 156 (42), 142 (69), 128 (5), 126 (4), 114 (5), 110 (5), 102 (14), 88 (24), 82 (7), 70 (16), 59 (13), 55 (100), 42 (13).

### EXAMPLE 12

### N,N-bis(pyridin-3-ylmethyl)acetamide

A solution of 3,3-dipicolylamine (1.2 g, 6.0 mmol) and Et₃N (1.0 ml, 7.2 mmol) in dichloromethane (30 ml) was treated with acetyl chloride (0.52 g, 6.6 mmol). The resulting mixture was stirred for 1 h, poured into 2N aq. NaOH solution (20 ml) and extracted twice with AcOEt (30 ml). The combined organic phases were washed with water (20 ml), with aqueous NaCl solution (20 ml), dried (MgSO₄), and the solvent evaporated. FC (50 g SiO₂, methyl t-butyl ether/MeOH 2:1) of the crude product (1.6 g) gave N,N-bis(pyridin-3-ylmethyl)acetamide (0.42 g, 29%). Boiling point: 205° C. (0.07 mbar).

¹³C-NMR (100 MHz, CDCl₃): δ 170.94 (s), 149.48 (d), 149.35 (d), 148.97 (d), 148.37 (d), 136.29 (d), 134.11 (d), 132.69 (s), 131.56 (s), 123.81 (d), 123.71 (d), 48.97 (t), 45.89 (t), 21.60 (q).

MS (EI): 241 (3), 226 (1), 198 (1), 149 (29), 121 (5), 107 (100), 93 (79), 92 (31), 80 (8), 65 (27), 43 (23), 39 (13).

### EXAMPLE 13

### methyl bis(pyridin-3-ylmethyl)carbamate

At 0° C., a solution of 3,3-dipicolylamine (1.0 g, 4.9 mmol) and Et₃N (0.6 g, 5.8 mmol) in dichloromethane (15 ml) was treated with a solution of methyl chloroformate (0.5 g, 5.4 mmol) in dichloromethane (5 ml). The resulting mixture was stirred at 20° C. for 24 h, poured into 2N aq. NaOH solution and extracted three times with AcOEt (80 ml). The combined organic phases were washed with water (80 ml), with aqueous NaCl solution (80 ml), dried (MgSO₄), and the solvent evaporated. FC (60 g SiO₂, methyl t-butyl ether/MeOH 10:1) of the crude product (0.8 g) gave methyl bis(pyridin-3-ylmethyl)carbamate (0.74 g, 57%). Boiling point: 200° C. (0.09 mbar).

¹³C-NMR (100 MHz, CDCl₃): δ 157.04 (s), 149.06 (d, 2C), 135.98, 135.30 (br. d, 1C), 132.63 (s), 123.63 (d), 53.26 (q), 47.58, 47.32 (br. t, 2C).

MS (EI): 257 (8), 242 (1), 226 (2), 165 (52), 133 (9), 122 (13), 121 (23), 93 (100), 92 (86), 78 (9), 65 (52), 59 (18), 51 (9), 42 (12), 39 (20).

### EXAMPLE 14

### N,N-bis(pyridin-2-ylmethyl)acetamide

Prepared in 55% yield after FC (SiO₂, methyl t-butyl ether/methanol 13:1) by acetylation of 2,2-dipicolylamine as described in Example 12. Boiling point: 185° C. (0.07 mbar).

¹³C-NMR (100 MHz, CDCl₃): δ 171.59 (s), 157.31 (s), 156.67 (s), 149.88 (d), 148.97 (d), 136.81 (d), 136.80 (d), 122.62 (d), 122.44 (d), 122.29 (d), 120.75 (d), 53.92 (t), 51.11 (t), 21.69 (q).

MS (EI): 241 (1), 226 (1), 198 (8), 181 (1), 171 (5), 149 (5), 121 (5), 107 (16), 93 (100), 92 (17), 78 (4), 65 (13), 43 (7).

### EXAMPLE 15

### methyl bis(pyridin-2-ylmethyl)carbamate

Prepared in 39% yield after FC (SiO₂, AcOEt) by acylation of 2,2-dipicolylamine as described in Example 13. Boiling point: 180° C. (0.07 mbar).

¹³C-NMR (100 MHz, CDCl₃): δ 157.53 (br. s), 157.40 (br. s), 157.35 (s), 149.39 (br. d), 149.27 (br. d), 136.62 (d, 2C), 122.21 (br. d), 122.10 (br. d), 121.97 (br. d), 120.94 (br. d), 53.02 (q), 52.63 (br. t), 52.22 (br. t).

MS (EI): 257 (1), 225 (3), 198 (1), 165 (7), 133 (14), 93 (100), 92 (16), 78 (5), 65 (14), 59 (3), 51 (4), 39 (4).

### EXAMPLE 16

### N-pentyl-N-(2-(pyridin-2-yl)ethyl)acetamide

Prepared as described in Example 1 from 2-(pyridin-2-yl)ethanamine and n-pentyliodide via N-(2-(pyridin-2-yl)ethyl)pentan-1-amine (20%, crude product). Acetylation gave after FC (SiO₂, methyl t-butyl ether) N-pentyl-N-(2-(pyridin-2-yl)ethyl)acetamide (30%). Boiling point: 160° C. (0.08 mbar).

¹³C-NMR (100 MHz, CDCl₃): δ (main rotamer, 55%) 170.26 (s), 159.33 (s), 149.60 (d), 136.61 (d), 123.73 (d), 121.43 (d), 49.43 (t), 46.22 (t), 36.26 (t), 28.88 (t), 28.62 (t), 22.36 (t), 21.50 (q), 13.91 (q). δ (minor rotamer, 45%) 170.23 (s), 158.26 (s), 148.96 (d), 136.61 (d), 123.51 (d), 121.75 (d), 48.35 (t), 45.51 (t), 37.45 (t), 29.13 (t), 27.34 (t), 22.45 (t), 21.37 (q), 13.97 (q).

MS (EI): 234 (9), 219 (1), 205 (3), 191 (5), 178 (9), 164 (3), 149 (8), 142 (5), 135 (26), 121 (13), 107 (24), 106 (100), 105 (15), 100 (62), 94 (29), 93 (30), 78 (13), 65 (6), 44 (27), 43 (41).

### EXAMPLE 17

### methyl pentyl(2-(pyridin-2-yl)ethyl)carbamate

Prepared as described in Example 16 from 2-(pyridin-2-yl)ethanamine and n-pentyliodide via N-(2-(pyridin-2-yl)ethyl)pentan-1-amine. Acylation using methyl chloroformate gave after FC (SiO₂, methyl t-butyl ether/hexane 35:65) methyl pentyl(2-(pyridin-2-yl)ethyl)carbamate (16%). Boiling point: 150° C. (0.08 mbar).

¹³C-NMR (100 MHz, CDCl₃): δ (rotameric mixture) 159.30 (s), 156.79 (s), 149.36 (br. d), 136.38 (d), 123.52 (br. d), 121.39 (d), 53.67 (br. t), 52.34 (q), 47.79 (br. t), 37.54, 36.94 (t, 1C), 28.90 (t), 28.34, 27.83 (br. t, 1C), 22.43 (t), 13.99 (q).

MS (EI): 250 (11), 235 (1), 221 (3), 219 (3), 207 (10), 205 (5), 194 (8), 193 (7), 191 (2), 180 (5), 170 (17), 158 (10), 147 (6), 107 (34), 106 (100), 105 (23), 102 (91), 93 (28), 88 (8), 78 (15), 65 (7), 59 (16), 43 (17).

### EXAMPLE 18

### N-pentyl-N-(2-(pyridin-3-yl)ethyl)acetamide

Prepared as described in Example 1 from 2-(pyridin-3-yl)ethanamine and n-pentyliodide via N-(2-(pyridin-3-yl)ethyl)pentan-1-amine (80%, crude product). Acetylation gave after FC (SiO₂, methyl t-butyl ether) N-pentyl-N-(2-(pyridin-3-yl)ethyl)acetamide (55%). Boiling point: 170° C. (0.07 mbar).

¹³C-NMR (100 MHz, CDCl₃): δ (main rotamer, 75%) 170.27 (s), 149.87 (d), 147.61 (d), 136.47 (d), 134.83 (s), 123.41 (d), 49.56 (t), 47.50 (t), 31.19 (t), 28.86 (t), 28.63 (t), 22.43 (t), 21.49 (q), 13.88 (q). δ (minor rotamer, 35%) 169.91 (s), 149.93 (d), 148.26 (d), 134.83 (d), 133.61 (s), 123.51 (d), 49.84 (t), 45.66 (t), 32.49 (t), 29.10 (t), 27.36 (t), 22.34 (t), 21.38 (q), 13.95 (q).

MS (EI): 234 (5), 219 (1), 191 (1), 177 (1), 142 (36), 135 (10), 106 (15), 100 (100), 94 (2), 93 (11), 44 (26), 43 (29).

### EXAMPLE 19

### methyl pentyl(2-(pyridin-3-yl)ethyl)carbamate

Prepared as described in Example 16 from 2-(pyridin-3-yl)ethanamine and n-pentyliodide via N-(2-(pyridin-3-yl)ethyl)pentan-1-amine. Acylation using methyl chloroformate gave after FC (SiO₂, methyl t-butyl ether/hexane 2:1) methyl pentyl(2-(pyridin-3-yl)ethyl)carbamate (40%). Boiling point: 160° C. (0.08 mbar).

¹³C-NMR (100 MHz, CDCl₃): δ (rotameric mixture) 156.65 (br. s), 150.08 (d), 147.77 (d), 136.27 (d), 134.52 (br. s), 123.31 (d), 52.39 (q), 49.06, 48.27 (br. t, 1C), 47.74 (br. t), 32.38, 31.70 (br. t, 1C), 28.84 (t), 28.28, 27.83 (br. t, 1C), 22.36 (t), 13.93 (q).

MS (EI): 250 (4), 235 (1), 193 (1), 161 (2), 158 (37), 106 (11), 102 (100), 93 (4), 88 (7), 59 (10), 43 (10).

### EXAMPLE 20

### N-pentyl-N-(2-(pyridin-4-yl)ethyl)acetamide

Prepared as described in Example 1 from 2-(pyridin-4-yl)ethanamine and n-pentyliodide via N-(2-(pyridin-4-yl)ethyl)pentan-1-amine (80%, crude product). Acetylation gave after FC (SiO₂, AcOEt/isopropanol 85:15) N-pentyl-N-(2-(pyridin-4-yl)ethyl)acetamide (55%). Boiling point: 170° C. (0.07 mbar).

¹³C-NMR (100 MHz, CDCl₃): δ (main rotamer, 70%) 170.24 (s), 149.65 (d, 2C), 148.46 (s), 124.16 (d, 2C), 49.51 (t), 46.83 (t), 33.39 (t), 28.83 (t), 28.60 (t), 22.31 (t), 21.48 (q), 13.86 (q). δ (minor rotamer, 30%) 169.89 (s), 150.07 (s), 147.09 (d, 2C), 123.98 (d, 2C), 49.15 (t), 45.62 (t), 34.65 (t), 29.07 (t), 27.32 (t), 22.40 (t), 21.35 (q), 13.93 (q).

MS (EI): 234 (7), 219 (1), 177 (2), 142 (35), 135 (18), 106 (18), 100 (100), 93 (12), 44 (26), 43 (31).

### EXAMPLE 21

### methyl pentyl(2-(pyridin-4-yl)ethyl)carbamate

Prepared as described in Example 16 from 3-(pyridin-4-yl)ethanamine and n-pentyliodide via N-(2-(pyridin-4-yl)ethyl)pentan-1-amine. Acylation using methyl chloroformate gave after FC (SiO₂, methyl t-butyl ether/hexane 2:1) methyl pentyl(2-(pyridin-4-yl)ethyl)carbamate (49%). Boiling point: 160° C. (0.07 mbar).

¹³C-NMR (100 MHz, CDCl₃): δ (rotameric mixture) 156.60 (br. s), 149.75 (d, 2C), 148.1 (br. s), 124.15 (d, 2C), 52.37 (q), 48.39, 47.74 (br. t, 1C), 47.60 (br. t), 34.56, 33.92 (br. t, 1C), 28.82 (t), 28.21, 27.83 (br. t, 1C), 22.34 (t), 13.91 (q).

MS (EI): 250 (6), 235 (1), 219 (1), 193 (4), 161 (2), 158 (37), 149 (2), 133 (1), 120 (2), 106 (10), 102 (100), 93 (4), 88 (7), 59 (12), 43 (10).

### EXAMPLE 22

### N-pentyl-N-(pyridin-2-ylmethyl)acetamide

Prepared as described in Example 1 from 2-(aminomethyl)pyridine and n-pentyliodide via N-(pyridin-2-ylmethyl)pentan-1-amine. Acetylation gave after FC (SiO₂, ethyl acetate) N-pentyl-N-(pyridin-2-ylmethyl)acetamide (39%). Boiling point: 140° C. (0.08 mbar).

¹³C-NMR (100 MHz, CDCl₃): δ (major rotamer, 63%) 170.53 (s), 157.91 (s), 148.81 (d), 136.78 (d), 122.34 (d), 122.18 (d), 50.64 (t), 49.10 (t), 28.87 (t), 28.22 (t), 22.32 (t), 21.31 (q), 13.88 (q). δ (minor rotamer, 37%) 170.99 (s), 157.35 (s), 149.78 (d), 136.96 (d), 122.49 (d), 120.17 (d), 53.99 (t), 46.47 (t), 29.06 (t), 27.23 (t), 22.39 (t), 21.81 (q), 13.92 (q).

MS (EI): 220 (1), 205 (1), 177 (3), 163 (1), 121 (16), 107 (4), 93 (100), 92 (12), 65 (6), 43 (8).

### EXAMPLE 23

### methyl pentyl(pyridin-2-ylmethyl)carbamate

Prepared as described in Example 4 from 2-(aminomethyl)pyridine and n-pentyliodide via N-(pyridin-2-ylmethyl)pentan-1-amine. Acylation using methyl chloroformate gave after FC (SiO₂, methyl t-butyl ether/hexane 1:2) methyl pentyl(pyridin-2-ylmethyl)carbamate (47%). Boiling point: 155° C. (0.08 mbar).

¹³C-NMR (100 MHz, CDCl₃): δ (two rotamers) 158.23 (br. s), 157.36 and 156.82 (br. s, 1 C), 149.16 and 149.01 (br. d, 1C), 136.71 (d), 122.12 (br. d), 121.82 and 120.72 (br. d, 1C), 52.62 (q), 52.56 and 52.19 (br. t, 1C), 47.83 and 47.21 (br. t, 1C), 28.82 (t), 27.82 and 27.45 (br. t, 1C), 22.32 (t), 13.90 (q).

MS (EI): 236 (1), 221 (1), 205 (1), 179 (5), 107 (6), 93 (100), 92 (16), 65 (7), 59 (3), 41 (3).

### EXAMPLE 27

### N-(2-(1H-imidazol-4-yl)ethyl)-N-pentylacetamide

The starting N-(2-(1H-imidazol-4-yl)ethyl)pentan-1-amine was prepared according to the literature (Young, R. C.; Ganellin, C. R.; Griffiths, R.; Mitchell, R. C.; Parsons, M. E.; Saunders, D.; Sore, N. E. Eur. J. Med. Chem. 1993, 28, 201-11) from histamine dihydrochloride by transformation into 7,8-dihydroimidazo[1,5-c]pyrimidin-5(6H)-one (carbonyldiimidazole in DMF), subsequent alkylation (n-pentyliodide, NaH, DMF) into 6-pentyl-7,8-dihydroimidazo[1,5-c]pyrimidin-5(6H)-one followed by hydrolysis using aqueous KOH.

Acetylation as described in Example 1 gave after FC (90 g SiO₂, AcOEt/isopropanol 9:1 to 1:1) of the reaction mixture N-(2-(1H-imidazol-4-yl)ethyl)-N-pentylacetamide (0.78 g, 70%). Boiling point: 220° C. (0.07 mbar).

¹³C-NMR (100 MHz, (CD₃)₂SO): δ (1:1 rotameric mixture) 169.55, 169.54 (s, 1C), 135.23, 134.99 (d, 1C), 134.45 (br. s, 1C), 117.43, 117.14 (br. d, 1C), 48.75, 48.44 (t, 1C), 45.75, 44.94 (t, 1C), 29.10, 28.86, 28.55, 27.37, 26.81, 25.51 (t, 3C), 22.38, 22.32, 21.76, 21.58 (t, 2C), 14.35 (q).

MS (EI): 223 (6), 180 (14), 142 (11), 130 (31), 124 (12), 100 (100), 95 (34), 94 (69), 82 (25), 81 (20), 68 (8), 54 (9), 44 (34), 43 (43).

### EXAMPLE 28

### methyl 2-(1H-imidazol-4-yl)ethyl(pentyl)carbamate

Acylation as described in Example 16 of N-(2-(1H-imidazol-4-yl)ethyl)pentan-1-amine (prepared as described in Example 27) using methyl chloroformate gave after FC (90 g SiO₂, AcOEt/isopropanol 9:1 to 1:1) of the reaction mixture methyl 2-(1H-imidazol-4-yl)ethyl(pentyl)carbamate (0.89 g, 75%). Boiling point: 200° C. (0.07 mbar).

¹³C-NMR (100 MHz, (CD₃)₂SO): δ (rotameric mixture) 156.24 (s, 1C), 138.22 (br. s), 135.10 (d), 112.77 (br. d), 52.50 (q), 47.35 (br. t), 47.06 (br. t), 28.83 (t), 28.21 (br. t), 27.72 (br. t), 22.31 (t), 14.31 (q).

MS (EI): 239 (5), 208 (1), 196 (3), 182 (3), 180 (3), 158 (17), 102 (100), 95 (20), 94 (40), 88 (8), 81 (11), 68 (5), 59 (12), 43 (12).

### EXAMPLE 29

### methyl benzyl(pentyl)carbamate

Prepared as described in Example 4 from benzylamine and n-pentyliodide via N-benzyl-N-pentylamine. Acylation using methyl chloroformate gave after FC (SiO₂, methyl t-butyl ether/hexane 1:10) methyl benzyl(pentyl)carbamate (81%). Boiling point: 125° C. (0.09 mbar).

¹³C-NMR (100 MHz, CDCl₃): δ (two rotamers) 157.40 and 156.93 (2 br. s, 1C), 138.06 (s), 128.47 (d, 2C), 127.78 (br. d), 126.19 (d, 2C), 52.54 (q), 50.37 and 50.02 (2t, 1C), 47.00 and 46.20 (2t, 1C), 28.89 (t), 27.64 and 27.37 (2t, 1C), 22.35 (t), 13.92 (q).

MS (EI): 235 (8), 179 (4), 178 (40), 92 (8), 91 (100), 88 (3), 65 (7), 59 (2), 42 (2), 41 (3).

### EXAMPLE 30

### 1,1-dimethyl-3-pentyl-3-(pyridin-3-ylmethyl)urea

Prepared as described in Example 24 from 3-(aminomethyl)pyridine and n-pentyliodide via N-(pyridin-3-ylmethyl)pentan-1-amine (prepared as described in Example 1, 58%, after ball-to-ball distillation at 120° C., 0.06 mbar of the crude product). Acylation using dimethylcarbamoyl chloride gave after FC (150 g SiO₂, AcOEt) of the crude product (2.53 g) 1,1-dimethyl-3-pentyl-3-(pyridin-3-ylmethyl)urea (0.77 g, 30%). Boiling point: 170° C. (0.09 mbar).

¹³C-NMR (100 MHz, CDCl₃): δ 165.20 (s), 149.28 (d), 148.44 (d), 135.58 (d), 134.13 (s), 123.37 (d), 48.82 (t), 48.72 (t), 38.57 (q, 2C), 28.94 (t), 27.25 (t), 22.31 (t), 13.88 (q).

MS (EI): 249 (9), 205 (4), 192 (17), 178 (6), 177 (10), 157 (6), 135 (5), 119 (4), 107 (3), 92 (47), 72 (100), 65 (13), 44 (5).

### EXAMPLE 31

### 3-methyl-1-pentyl-1-(pyridin-3-ylmethyl)urea

Prepared as described in Example 25 from 3-(aminomethyl)pyridine and n-pentyliodide via N-(pyridin-3-ylmethyl)pentan-1-amine (prepared as described in Example 1, 58%, after ball-to-ball distillation at 120° C., 0.06 mbar of the crude product). Acylation using N,N′-dimethylurea gave after FC (90 g SiO₂, AcOEt/isopropanol 1:o to 1:1) of the reaction mixture 3-methyl-1-pentyl-1-(pyridin-3-ylmethyl)urea (0.84 g, 71%). Boiling point: 200° C. (0.08 mbar).

¹³C-NMR (100 MHz, CD₃SOCD₃): δ 158.48 (s), 149.17 (d), 148.47 (d), 135.43 (s), 135.39 (d), 123.85 (d), 47.32 (t), 46.53 (t), 28.84 (t), 27.97 (t), 27.78 (q), 22.38 (t), 14.33 (q).

MS (EI): 235 (9), 178 (6), 121 (100), 107 (16), 93 (17), 92 (96), 80 (5), 65 (21), 57 (16).

### EXAMPLE 32

### 1-pentyl-1-(pyridin-3-ylmethyl)urea

Prepared as described in Example 26 from 3-(aminomethyl)pyridine and n-pentyliodide via N-(pyridin-3-ylmethyl)pentan-1-amine (prepared as described in Example 1, 58%, after ball-to-ball distillation at 120° C., 0.06 mbar of the crude product). Acylation using urea gave after FC (90 g SiO₂, methyl t-butyl ether/AcOEt 10:0 to 0:10) of the reaction mixture 1-pentyl-1-(pyridin-3-ylmethyl)urea (0.65 g, 58%). Boiling point: 220° C. (0.08 mbar).

¹³C-NMR (100 MHz, (CD₃)₂SO): δ 158.83 (s), 149.18 (d), 148.48 (d), 135.40 (d and s, 2 C), 123.84 (d), 47.25 (t), 46.76 (t), 28.81 (t), 27.92 (t), 22.39 (t), 14.33 (q).

MS (EI): 221 (4), 164 (3), 122 (7), 121 (86), 107 (13), 93 (13), 92 (100), 80 (5), 65 (20), 43 (13).

### EXAMPLE 33

### N-acetyl-N-pentylcyclopropanecarboxamide

A solution of N-(n-pentyl)acetamide (2.0 g, 15.5 mmol) in pyridine (1.4 g, 17 mmol) and 1,2-dichloroethane (20 ml) was treated dropwise with cyclopropanecarbonyl chloride (1.8 g, 17 mmol) and the resulting mixture was heated at reflux for 2 h, cooled, poured into ice-cold 2M aq. HCl (50 ml), and extracted twice with MTBE (80 ml). The combined organic phases were washed with water (80 ml), with a saturated aqueous NaCl solution (80 ml), dried (MgSO₄) and the solvent evaporated. FC (150 g SiO₂, hexane/methyl t-butyl ether 8:1) of the crude product (4.2 g) gave N-acetyl-N-pentylcyclopropanecarboxamide (1.4 g, 46%). Boiling point: 137° C. (0.08 mbar).

¹³C-NMR (100 MHz, CDCl₃): δ 177.19 (s), 173.14 (s), 49.54 (t), 29.03 (t), 28.97 (t), 26.58 (q), 22.28 (t), 15.04 (d), 13.89 (q), 10.27 (t, 2C).

MS (EI): 197 (1), 182 (5), 169 (13), 154 (3), 140 (2), 128 (7), 112 (57), 98 (10), 86 (10), 69 (100), 55 (5), 43 (37), 41 (38).

### EXAMPLE 34

### tert-butyl acetyl(pentyl)carbamate

A solution of N-(n-pentyl)acetamide (2.0 g, 15.5 mmol) in dichloromethane (50 ml) was treated with Et₃N (1.6 g, 15.5 mmol), di-tert-butyl dicarbonate (6.8 g, 31 mmol), and DMAP (1.9 g, 15.5 mmol). The resulting mixture was stirred for 24 h at 20° C. and the solvent evaporated. FC (160 g SiO₂, hexane/methyl t-butyl ether 10:1) of the crude product (5.3 g) gave tert-butyl acetyl(pentyl)carbamate (0.3 g, 9%). Boiling point: 80° C. (0.08 mbar).

¹³C-NMR (100 MHz, CDCl₃): δ 172.90 (s), 153.90 (s), 82.64 (s), 44.25 (t), 29.04 (t), 28.28 (t), 28.00 (q), 26.87 (q), 22.32 (t), 13.94 (q).

MS (EI): 229 (1), 214 (1), 173 (2), 158 (7), 130 (4), 114 (5), 101 (3), 100 (4), 87 (6), 86 (7), 73 (11), 72 (13), 57 (100), 44 (18), 43 (38), 41 (31), 30 (17).

### EXAMPLE 35

### N-benzyl-N-phenethylacetamide

At 20° C., a mixture of N-acetyl-2-phenylethylamine (2 g, 12.2 mmol) and NaH (0.51 g, 13.5 mmol) in dimethoxyethane (20 ml) was treated with a solution of benzyl bromide (3.1 g, 18.3 mmol) in DME (10 ml). The resulting mixture was stirred for 3.5 h poured into ice-cold 2M aq. HCl (50 ml), and extracted twice with MTBE (80 ml). The combined organic phases were washed with water (80 ml), with a saturated aqueous NaCl solution (80 ml), dried (MgSO₄) and the solvent evaporated. FC (90 g SiO₂, hexane/methyl t-butyl ether 1:1) of the crude product (4.25 g) gave N-benzyl-N-phenethylacetamide (2.39 g, 77%). Boiling point: 202° C. (0.06 mbar).

¹³C-NMR (100 MHz, CDCl₃): δ (1:1 rotameric mixture) 170.93 (s), 170.68 (s), 139.25 (s), 138.18 (s), 137.61 (s), 136.81 (s), 128.90, 128.83, 128.76, 128.71, 128.60, 128.48, 128.14, 127.59, 127.38, 126.76, 126.31 (d, 10C), 52.70 (t), 49.45 (t), 48.21 (t), 48.11 (t), 34.88 (t), 33.98 (t), 21.83 (q), 21.22 (q).

MS (EI): 253 (8), 238 (1), 162 (29), 120 (90), 104 (6), 91 (100), 77 (5), 65 (15), 44 (1).

### EXAMPLE 36

### N-(cyclopropylmethyl)-N-pentylformamide

A mixture of N-(n-pentyl)formamide (1.0 g, 8.7 mmol), and NaH (0.417 g, 9.6 mmol) in DME (15 ml) was treated dropwise with a solution of bromomethylcyclopropane (1.83 g, 13.0 mmol) in DME (10 ml) and the resulting mixture was heated at 60° C. for 22 h, cooled to 0° C., poured into ice-cold 2M aq. HCl (30 ml), and extracted twice with MTBE (50 ml). The combined organic phases were washed with water (50 ml), with a saturated aqueous NaCl solution (50 ml), dried (MgSO₄) and the solvent evaporated. FC (50 g SiO₂, hexane/methyl t-butyl ether 1:1) of the crude product (1.64 g) gave N-(cyclopropylmethyl)-N-pentylformamide (1.04 g, 68%). Boiling point: 80° C. (0.05 mbar).

¹³C-NMR (100 MHz, CDCl₃): δ (1:1 rotamer mixture) 162.64 (d), 162.25 (d), 52.39 (t), 47.32 (t), 46.06 (t), 42.51 (t), 29.02 (t), 28.55 (t), 28.18 (t), 26.87 (t), 22.31 (t), 22.18 (t), 13.89 (q), 13.84 (q), 10.47 (d), 9.30 (d), 3.81 (t), 3.66 (t).

MS (EI): 169 (6), 168 (5), 154 (6), 140 (33), 126 (14), 112 (46), 98 (16), 84 (18), 82 (10), 72 (15), 71 (19), 70 (10), 58 (17), 56 (24), 55 (100), 44 (12), 43 (22), 42 (11), 41 (19), 39 (13), 29 (17).

### EXAMPLE 37

### (E)-2-benzylidene-N-methylheptanamide

At 5° C., a solution of methylamine hydrochloride (1.35 g, 20 mmol) in benzene (20 ml) was treated dropwise within 20 min. with a 2M solution of trimethylaluminium in toluene (10 ml, 20 mmol) and the resulting mixture was then stirred for 1.5 h at 20° C. A part of the resulting solution (26 ml, 17.2 mmol) was added dropwise to a solution of (E)-methyl 2-benzylideneheptanoate (2 g, 0.86 mmol, prepared as described in Example 38 in 48% yield from methyl heptanoate and benzaldehyde) in benzene (40 ml) and the reaction mixture was then refluxed for 5 h, cooled, poured into ice-cold 2M aqueous HCl (80 ml), and extracted twice with ethyl acetate (80 ml). The combined organic phases were washed with water (80 ml), aqueous NaCl solution (80 ml), dried (MgSO₄), and the solvent evaporated. FC (150 g SiO₂, hexane/methyl t-butyl ether 1:1) of the crude product (2.26 g) gave (E)-2-benzylidene-N-methylheptanamide (1.46 g, 73%). Boiling point: 170° C. (0.09 mbar).

¹³C-NMR (100 MHz, CDCl₃): δ 170.71 (s, C(1)), 139.07 (s), 136.15 (s), 131.99 (d), 128.82 (d, 2C), 128.35 (d, 2C), 127.65 (d), 31.84 (t), 28.65 (t), 27.97 (t), 26.63 (q, MeN), 22.37 (t), 13.97 (q, C(7)).

MS (EI): 232 (15), 231 (90), 230 (43), 202 (53), 201 (20), 189 (18), 188 (21), 175 (31), 174 (58), 160 (20), 145 (24), 143 (19), 140 (16), 131 (41), 130 (26), 129 (33), 128 (28), 120 (64), 117 (99), 116 (37), 115 (100), 103 (11), 91 (90), 77 (15), 58 (88).

### EXAMPLE 38

### (E)-2-(cyclopropylmethylene)-N-methylheptanamide

a) At −75° C., a solution of diisopropylamine (7.7 ml, 54.1 mmol) in tetrahydrofuran (60 ml) was treated with a 1.6M solution of n-butyllithium in hexane (34 ml, 54.1 mmol). The resulting solution was stirred for 30 min. at −75° C. and treated with a solution of methyl heptanoate (6.0 g, 41.6 mmol) in tetrahydrofuran (20 ml). The resulting solution was stirred for 30 min. at −75° C. and treated with a solution of cyclopropanecarboxaldehyde (12.7 ml, 166.4 mmol) in tetrahydrofuran (20 ml). After stirring for 2 h at −75° C., the reaction mixture was poured into ice-cold 2M aqueous HCl (50 ml) and extracted twice with methyl t-butyl ether (100 ml). The combined organic phases were washed with water (50 ml), aqueous NaCl solution (50 ml), dried (MgSO₄), and the solvent evaporated to give an oil (9.66 g). A part of this residue (4.83 g) was treated with acetic anhydride (4.5 ml, 47.3 mmol) and sodium acetate (2.04 g, 24.8 mmol). The resulting mixture was stirred for 32 h at 80° C. and for 65 h at 20° C., poured into an ice-cold 2M NaOH solution (50 ml) and extracted twice with methyl t-butyl ether (50 ml). The combined organic phases were washed with a saturated aqueous solution of NaHCO₃ (25 ml), water (25 ml), aqueous NaCl solution (25 ml), dried (MgSO₄), and the solvent evaporated to give an oil (5.28 g). A solution of a part of this residue (2.6 g) in toluene (20 ml) was treated at 20° C. with a solution of DBU (3.1 ml, 20.3 mmol) in toluene (5 ml). The resulting solution was stirred for 1 h at 20° C., 1 h at 50° C. and 28 h at reflux, poured into ice-cold 2M aqueous HCl (50 ml), and extracted twice with methyl t-butyl ether (50 ml). The combined organic phases were washed with water (50 ml), aqueous NaCl solution (50 ml), dried (MgSO₄), and the solvent evaporated. FC (100 g SiO₂, hexane/methyl t-butyl ether 60:1) of the crude product (2.1 g) gave (E)-methyl 2-(cyclopropylmethylene)heptanoate (0.6 g, 29%). Boiling point: 95° C. (0.07 mbar).

¹H-NMR (400 MHz, CDCl₃): δ6.10 (d, J=10.6, H—C═C(2)), 3.71 (s, OMe), 2.39 (br. t, J=7.7, 2 H—C(3)), 1.67-1.57 (m, H—CCH═), 1.50-1.25 (m, C(4)H₂, C(5)H₂, C(6)H₂), 0.94 (ddd, J=4.3, 6.6, 7.8, 2 H), 0.89 (t, J=7.1, C(7)H₃), 0.59 (dt, J=4.5, 6.8, 2 H).

¹³C-NMR (100 MHz, CDCl₃): δ 168.37 (s, C(1)), 147.65 (d, CH═C(2)), 130.03 (s, C(2)), 51.44 (q, OMe), 31.69 (t), 29.14 (t), 26.82 (t), 22.52 (t), 14.02 (q, C(7)), 11.42 (d), 8.35 (t, 2C).

MS (EI): 196 (12), 181 (21), 168 (90), 165 (15), 153 (3), 139 (20), 125 (30), 111 (65), 107 (38), 95 (30), 93 (27), 91 (15), 81 (44), 79 (100), 77 (37), 67 (50), 59 (33), 55 (37), 53 (24), 41 (41).

b) Treatment as described in Example 37 of (E)-methyl 2-(cyclopropylmethylene)-heptanoate with a mixture of methylamine hydrochloride in benzene and a 2M solution of trimethylaluminium in toluene led to (E)-2-(cyclopropylmethylene)-N-methylheptanamide in 72% yield. Boiling point: 145° C. (0.07 mbar).

¹³C-NMR (100 MHz, CDCl₃): δ 169.78 (s, C(1)), 140.45 (d, CH═C(2)), 133.97 (s, C(2)), 31.72 (t), 29.00 (t), 27.33 (t), 26.49 (q, MeN), 22.51 (t), 14.00 (q, C(7)), 10.75 (d), 7.80 (t, 2C).

MS (EI): 195 (4), 180 (12), 167 (53), 165 (8), 152 (9), 139 (30), 138 (54), 124 (19), 110 (100), 95 (31), 81 (75), 79 (38), 77 (23), 67 (47), 58 (97).

### EXAMPLE 39

### (E)-2-benzylidene-N,N-dimethylheptanamide

Prepared as described in Example 37 from (E)-methyl 2-benzylideneheptanoate and dimethylamine hydrochloride in 64% yield. Boiling point: 125° C. (0.09 mbar).

¹³C-NMR (100 MHz, CDCl₃): δ 173.06 (s, C(1)), 138.63 (s), 136.12 (s), 128.97 (d), 128.71 (d, 2C), 128.31 (d, 2C), 127.28 (d), 38.85 (br. q, MeN), 34.84 (br. q, MeN), 31.95 (t), 29.61 (t), 27.87 (t), 22.39 (t), 13.98 (q, C(7)).

MS (EI): 245 (39), 230 (4), 216 (11), 202 (21), 201 (72), 189 (20), 188 (37), 174 (14), 173 (4), 143 (8), 131 (19), 130 (23), 129 (20), 128 (18), 117 (100), 116 (23), 115 (65), 105 (14), 91 (95), 72 (51), 46 (6), 44 (9).

### EXAMPLE 40

### (E)-2-(cyclopropylmethylene)-N,N-dimethylheptanamide

Prepared as described in Example 38 from (E)-methyl 2-(cyclopropylmethylene)-heptanoate and dimethylamine hydrochloride in 85% yield. Boiling point: 105° C. (0.09 mbar).

¹³C-NMR (100 MHz, CDCl₃): δ 173.40 (s, C(1)), 135.48 (d, CH═C(2)), 134.29 (s, C(2)), 42.00-32.00 (br. q, Me₂N), 31.86 (t), 29.05 (t), 28.24 (t), 22.46 (t), 14.00 (q, C(7)), 9.86 (d), 6.98 (t, 2C).

MS (EI): 209 (9), 194 (6), 181 (52), 165 (58), 152 (46), 138 (9), 124 (60), 109 (25), 108 (17), 107 (19), 95 (40), 81 (86), 79 (52), 72 (100), 67 (71), 55 (39), 53 (21), 46 (15), 44 (15), 41 (39).

### EXAMPLE 41

### (E)-2-(cyclopropylmethylene)heptanamide

Prepared as described in Example 38 from (E)-methyl 2-(cyclopropylmethylene)-heptanoate and ammonium chloride in 89% yield. Boiling point: 150° C. (1.2 mbar).

¹³C-NMR (100 MHz, CDCl₃): δ 171.09 (s, C(1)), 142.71 (d, CH═C(2)), 132.74 (s, C(2)), 31.71 (t), 28.98 (t), 27.33 (t), 22.51 (t), 14.00 (q, C(7)), 11.02 (d), 8.04 (t, 2C).

MS (EI): 181 (4), 166 (19), 153 (77), 138 (9), 125 (29), 124 (51), 110 (34), 96 (100), 81 (94), 79 (49), 77 (28), 67 (58), 55 (28), 53 (29), 44 (36), 41 (41).

### EXAMPLE 42

### Evaluation of the Test Compounds as Inhibitors or CYP2A13

Compounds that inhibit the activity of CYP2A13 are identified by using a standard reaction established for the enzyme. A known substrate is coumarin, and the product of the enzymatic reaction is 7-hydroxy-coumarin (Umbelliferone) which is strongly fluorescent. When a compound is added to the standard reaction and the formation of Umbelliferone is decreased, the compound is identified as an inhibitor, which can also be a competitive substrate of the enzyme. The compound is used at various concentrations and the concentration-dependent decrease in Umbelliferone formation allows to determine the concentration where the activity of the enzyme is reduced to the 50% level (IC50 value).

A test compound (details see Table 1) was incubated with CYP2A13 in the presence of a cytochrome P450 reductase. CYP2A13 and P450 reductase were employed in form of microsomes. CYP2A13 was produced in Sf9 cells using a recombinant baculovirus, under conditions known to the person skilled in the art, for example, as described in WO 2006/007751. P450 reductase is commercially available (BD Biosciences Gentest, USA). Preferably, the two enzymes are coexpressed in the same insect cells and microsomes prepared which contain both enzymes. The art of coexpression of two enzymes is known, and the coexpression CYP2A13 and P450 reductase is described in WO 2006/007751. Variability of activity was observed for high-titer recombinant virus batches, and optimal multiplicity of infection (MOI) has to be determined as known to the skilled person. An MOI of 4 for recombinant CYP2A13 baculovirus combined with an MOI of 3.5 for recombinant P450 reductase baculovirus routinely produced microsomes with considerable activity.

Microsomes were used which contained 7 pmoles CYP2A13. Tris buffer (1 M, pH 7.6) and water were added to give a buffer concentration of 0.1M. The test compound was prepared as a 50 mM stock solution in acetonitrile. The concentration of the standard substrate coumarin was 0.006 mM. Several samples of the test compound were prepared at various concentrations to give different final concentrations in the reaction: 0, 0.005, 0.01, 0.02, 0.05, 0.1 and 0.2 mM. The mixture was incubated for 10 min at 37° C. prior to the initiation of the enzymatic reaction by the addition of 0.005 ml of a solution of 50 mM NADPH in water. The final total volume was 0.2 ml, which is suitable for microtiter plate measurements. The samples were incubated for 60 min at 37° C. After 60 min, the enzymatic reaction was stopped by the addition of 0.02 ml cold 50% trichloroacetic acid (TCA) and incubated at 4° C. for 15 min. 0.005 ml of a solution of 50 mM NADPH in water was added to the control reaction which corresponds to the reaction without test compound and without NADPH, and as a consequence, no Umbelliferone was formed. Denatured proteins and other insoluble parts were separated by centrifugation (10 min, 560×g, room-temperature).

The samples were analysed spectrofluorometrically which allows to detect the formation of Umbelliferone as the enzymatic product of coumarin at an excitation wavelength of 340 nm and an emission wavelength of 480 nm. A decrease of the fluorescent signal at 480 nm with respect to the control shows that the test compound is influencing enzymatic activity and confirms the nature of an inhibitor, since no metabolites have been detected. Graphical analysis of the data allows to calculate the concentration, where the test compound inhibits the enzyme to the level of 50% maximal activity (IC50 value).

TABLE 1
CYP2A13 inhibitor activity

| Compound | |
|---|---|
| IC₅₀ (μM) | Chemical Structure |
| Ex. 1 0.3 μM | 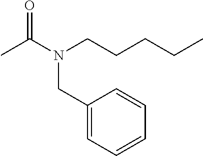 |
| Ex. 2 0.5 μM | 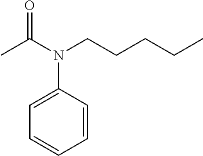 |
| Ex. 3 2 μM | 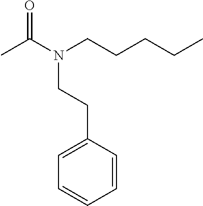 |
| Ex. 4 15 μM | 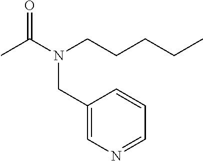 |
| Ex. 5 22 μM | 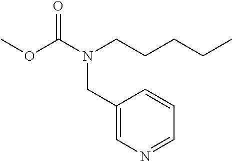 |
| Ex. 6 8 μM | 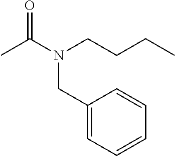 |
| Ex. 8 11 μM | 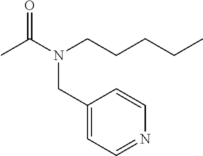 |
| Ex. 9 17 μM | 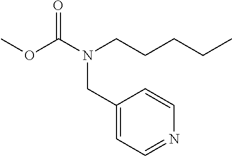 |
| Ex. 10 0.5 μM | 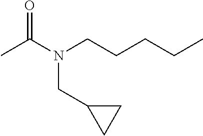 |
| Ex. 11 3 μM | 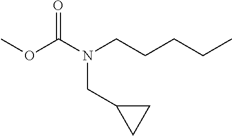 |
| Ex. 16 24 μM | 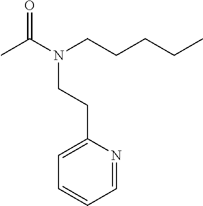 |
| Ex. 17 44 μM | 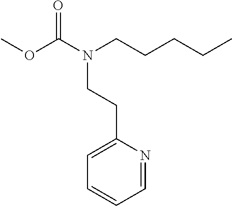 |
| Ex. 18 25 μM | 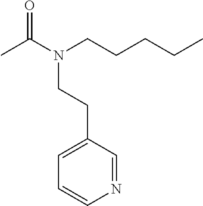 |
| Ex. 19 41 μM | 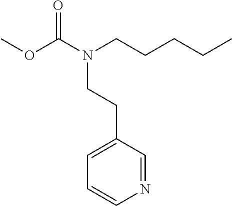 |
| Ex. 20 13 μM | 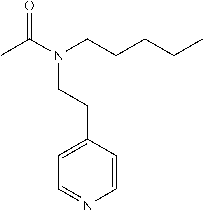 |
| Ex. 23 20 μM | 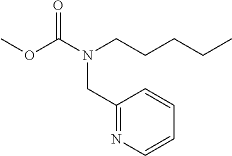 |
| Ex. 27 34 μM | 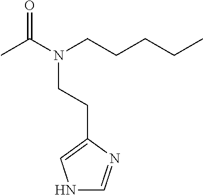 |
| Ex. 28 47 μM | 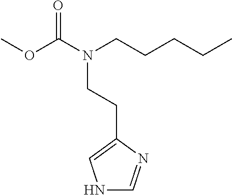 |
| Ex. 29 1.6 μM | 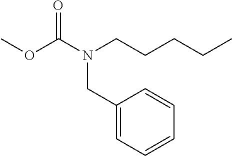 |
| Ex. 33 0.7 μM | 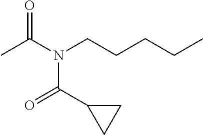 |
| Ex. 34 1.5 μM | 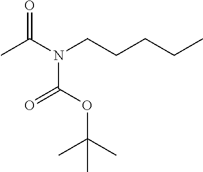 |
| Ex. 35 52 μM | 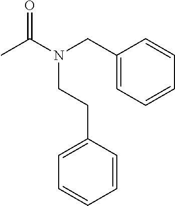 |
| Ex. 36 1.3 μM | 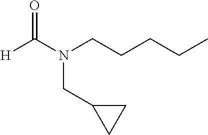 |
| Ex. 37 0.5 μM | 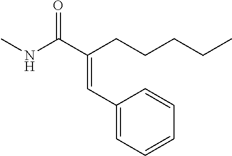 |
| Ex. 38 0.8 μM | 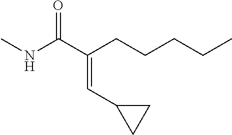 |
| Ex. 39 3 μM | 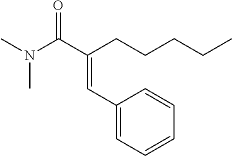 |
| Ex. 40 9 μM | 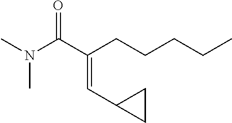 |
| Ex. 41 1.1 μM | 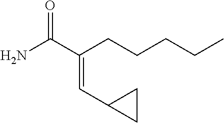 |
| Ex. 21 2 μM | 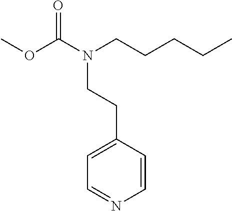 |
| Ex. 22 10 μM | 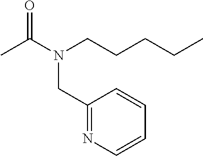 |

### EXAMPLE 43

### Evaluation of the Test Compounds as Inhibitors of CYP2B6

Test compounds that inhibit the activity of CYP2B6 are identified by using the same principle as described in Example 42, first paragraph.

A test compound (details see Table 2) was incubated with CYP2B6 in the presence of a cytochrome P450 reductase. CYP2B6 and P450 reductase are produced using recombinant baculoviruses and co-expressing the two proteins in Sf9 insect cells as described in Example 42. Alternatively, microsomes containing CYP2B6 and the reductase are commercially available (BD Biosciences Gentest, USA). Microsomes were used which contained 1.5 pmoles CYP2B6. Potassium phosphate buffer final concentration was 100 mM, (1M stock, pH 7.4). The test compound was prepared as a 50 mM stock solution in acetonitrile. The concentration of the standard substrate 7-ethoxy-4-trifluoromethyl-coumarin was 6 μM. Several samples of the test compound were prepared at various concentrations to give different final concentrations in the reaction: 0, 0.005, 0.01, 0.02, 0.05, 0.1 and 0.2 mM. (As obvious to the person skilled in the art, in cases where very good inhibitors were tested, lower concentrations were also used in order to have concentrations above and below the IC50 concentration present in the test wells.) The mixture was incubated for 10 min at 37° C. prior to the initiation of the enzymatic reaction by the addition of 0.005 ml of a solution of 50 mM NADPH in water. The final total volume was 0.2 ml, which is suitable for microtiter plate measurements. The samples were incubated for 40 min at 37° C. After 40 min, the enzymatic reaction was stopped by the addition of 750 of 0.5M Tris-base/acetonitrile (18:72). 0.005 ml of a solution of 50 mM NADPH in water was added to the control reaction which corresponds to the reaction with test compound and enzyme but without NADPH, and as a consequence, no 4-trifluoromethyl-umbelliferone was formed. Denatured proteins and other insoluble parts were separated by centrifugation (5 min, 1800 rpm, at 10° C.).

The samples were analysed spectrofluorometrically which allows to detect the formation of 4-trifluoromethyl-umbelliferone as the enzymatic product at an excitation wavelength of 410 nm and an emission wavelength of 510 nm. A decrease of the fluorescent signal at 510 nm with respect to the control shows that the test compound is influencing enzymatic activity and confirms the nature of an inhibitor, which can also be an alternative substrate. Graphical analysis of the data allows to calculate the concentration, where the test compound inhibits the enzyme to the level of 50% maximal activity (IC50 value). The results are shown in Table 2 below.

TABLE 2
CYP2B6 inhibitor activity

| Compound | |
|---|---|
| IC₅₀ (μM) | Chemical Structure |
| Ex. 1 72 μM | 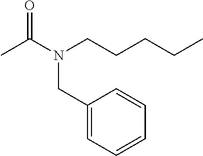 |
| Ex. 2 46 μM | 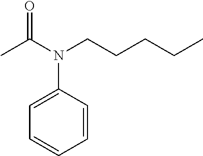 |
| Ex. 3 93 μM | 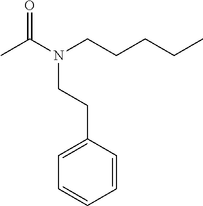 |
| Ex. 10 87 μM | 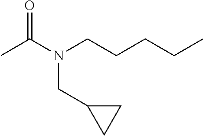 |
| Ex. 11 28 μM | 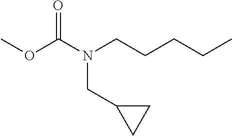 |
| Ex. 20 19 μM | 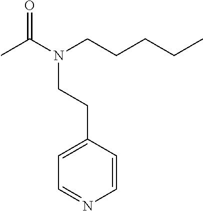 |
| Ex. 27 2.5 μM | 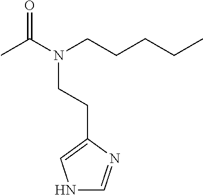 |
| Ex. 29 20 μM | 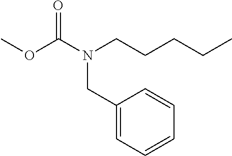 |
| Ex. 33 17 μM | 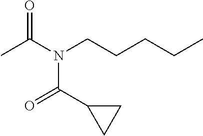 |
| Ex. 34 11 μM | 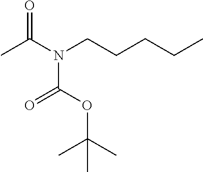 |
| Ex. 36 49 μM | 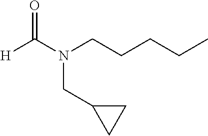 |

### EXAMPLE 44

### Modulation of an Odorant Compound in the Presence of a CYP2A Inhibitor

A fragrance accord consisting of 10 ingredients which have been selected in order to demonstrate an odor-modulating effect by the inhibitor was created. For each panelist, the inhibitor was tested by itself and confirmed that it was rated as being odorless at the given concentration.

### Fragrance Accord:

| | Parts by weight |
|---|---|
| | 1/900 |
| Benzyl-salicylate | 295 |
| Sandela ® (3-(5,5,6-trimethylbicyclo[2.2.1]hept-2-yl)- | 200 |
| cyclohexan-1-ol) | |
| Thibetolide (oxacyclohexadecan-2-one) | 140 |
| Super muguet (6-ethyl-3-methyl-6-octen-1-ol) | 90 |
| Epoxy cedrene (octahydro-3,6,6,7a-tetramethyl-2H-2a,7- | 70 |
| Methanoazuleno[5,6-b]oxirene) | |
| Eugenol | 50 |
| Grisalva (naphtho [2,1-b]-furan, 3a-ethyl dodecahydro- | 15 |
| 6,6,9a-trimethyl) | |
| Cis-3-hexenyl-acetate | 15 |
| Beta-damascone | 15 |
| Javanol ® (1-methyl-2-(1,2,2-trimethylbicyclo[3.1.0]- | 10 |
| hex-3-ylmethyl)cyclopropyl)methanol) | |

Sensory studies are performed using an olfactometer, such as the Virtual Aroma Synthesizer (VAS) which is described in Chimia (2001) 55:401-405. The instrument allows to combine saturated headspace of different samples from different containers at various dilutions in order to determine the effect on the odor of the mixture produced in headspace. For the particular example, in one container the fragrance accord (1 gram) was adsorbed on beads (4 grams) and in another container the inhibitor N-(cyclopropylmethyl)-N-pentylacetamide was adsorbed on beads (4 grams).

Panelists were selected having different levels of experience and expertise in smelling, rating, describing and evaluating odorants, accords and perfumes. Panelists smelled the accord with or without the inhibitor at random order, not knowing which one was presented. Before and after the session, it was confirmed that the inhibitor alone was odorless. Panelists were allowed to select a concentration of the accord that had a pleasant intensity.

The panelist reported an effect that was attributed to the presence of the inhibitor, independent of the experience with perfumery raw materials. The effect was described as intensifying or boosting the fruitiness of the accord.

In conclusion the example demonstrates that the use of an ingredient which has been identified as an inhibitor of the nasal CYP2A13 can modulate the olfactive quality of a fragrance accord.

1. A composition comprising
a) a compound of formula (I) 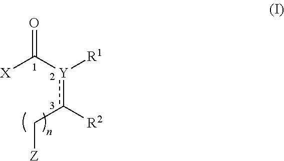
wherein n is 0 or 1;
R¹ is linear or branched C₃-C₇ alkyl, benzyl or pyridylmethyl;
R² is hydrogen, C₁-C₄ alkyl, or C₂-C₄ alkenyl; or
R² forms together with the carbon atom to which it is attached a carbonyl group;
I) Z is a 3-6 membered monocyclic or 6-10 membered bicyclic hydrocarbon ring wherein 0, 1 or 2, C atom(s) are replaced by a hetero atom selected from S, O, or N;
II) Z is a 3-6 membered monocyclic or 6-10 membered bicyclic hydrocarbon ring wherein 0, 1 or 2, C atom(s) are replaced by a hetero atom selected from S, O, or N, and the ring is substituted with up to 5 groups selected from hydroxyl, CN, halogen, mono-, di-, or trihalogenomethyl, C₁-C₃ alkoxy, C₁-C₃ alkyl, —COOR, or —OCOR wherein R is hydrogen, methyl, ethyl, propyl or isopropyl;
III) Z is a bivalent residue forming together with the C-3 a 3-6 membered monocyclic or 6-10 membered bicyclic hydrocarbon ring wherein 0, 1 or 2, C atom(s) are replaced by a hetero atom selected from S, O, or N;
IV) Z is a bivalent residue forming together with the C-3 a 3-6 membered monocyclic or 6-10 membered bicyclic hydrocarbon ring wherein 0, 1 or 2, C atom(s) are replaced by a hetero atom selected from S, O, or N, and the ring is substituted with up to 5 groups selected from hydroxyl, CN, halogen, mono-, di-, and or trihalogenomethyl, C₁-C₃ alkoxy, C₁-C₃ alkyl, —COOR, or —OCOR wherein R is hydrogen, methyl, ethyl, propyl or isopropyl; or
V) Z is C₁-C₄ alkoxy;
X is selected from hydrogen, C₁-C₃ alkyl, C₁-C₃ alkoxy, or NR³R⁴ wherein R³ and R⁴ independently are selected from hydrogen, or C₁-C₃ alkyl;
and Y represents a N- or C-atom with the proviso that
I) for X═NR³R⁴, Y represents a C-atom
II) for Y═C, the dotted line represents together with the carbon-carbon bond a double bond, either in E or Z configuration, or a single bond;
and
b) at least one odorant compound.

2. A composition according to claim 1 comprising a compound of formula (I) wherein
X is selected from hydrogen, methyl, and or methoxy, and
Y represents a N-atom.

3. A composition according to claim 1 comprising a compound of formula (I) wherein Z is selected from cyclopropyl, phenyl, pyridyl or imidazol.

4. A composition according to claim 1 wherein the compound of formula (I) is selected from the list consisting of N-benzyl-N-pentylacetamide, N-pentyl-N-phenylacetamide, N-butyl-N-phenylacetamide, N-pentyl-N-phenethylacetamide, N-pentyl-N-(pyridin-3-ylmethyl)acetamide, methyl pentyl(pyridin-3-ylmethyl)carbamate, N-benzyl-N-butylacetamide, methyl benzyl(butyl)carbamate, N-pentyl-N-(pyridin-4-ylmethyl)acetamide, methyl pentyl(pyridin-4-ylmethyl)carbamate, N-(cyclopropylmethyl)-N-pentylacetamide, methyl cyclopropylmethyl(pentyl)carbamate, N,N-bis(pyridin-3-ylmethyl)acetamide, methyl bis(pyridin-3-ylmethyl)carbamate, N,N-bis(pyridin-2-ylmethyl)acetamide, methyl bis(pyridin-2-ylmethyl)carbamate, N-pentyl-N-(2-(pyridin-2-yl)ethyl)acetamide, methyl pentyl(2-(pyridin-2-yl)ethyl)carbamate, N-pentyl-N-(2-(pyridin-3-yl)ethyl)acetamide, methyl pentyl(2-(pyridin-3-yl)ethyl)carbamate, N-pentyl-N-(2-(pyridin-4-yl)ethyl)acetamide, methyl pentyl(2-(pyridin-4-yl)ethyl)carbamate, N-pentyl-N-(pyridin-2-ylmethyl)acetamide, methyl pentyl(pyridin-2-ylmethyl)carbamate, N-(2-(1H-imidazol-4-yl)ethyl)-N-pentylacetamide, methyl 2-(1H-imidazol-4-yl)ethyl(pentyl)carbamate, methyl benzyl(pentyl)carbamate, N-acetyl-N-pentylcyclopropanecarboxamide, tert-butyl acetyl(pentyl)carbamate, N-benzyl-N-phenethylacetamide, N-(cyclopropylmethyl)-N-pentylformamide, (E)-2-benzylidene-N-methylheptanamide, (E)-2-benzylidene-N-methylheptanamide, (E)-2-benzylidene-N,N-dimethylheptanamide, (E)-2-(cyclopropylmethylene)-N,N-dimethylheptanamide, and (E)-2-(cyclopropylmethylene)heptanamide.

5. A tobacco product comprising a compound of formula (I) as defined in claim 1.

6. A method comprising the step of dissemination a compound of formula (I) as defined in claim 1, into a room in the presence of tobacco smoke.

7. A method according to claim 6 wherein the compound of formula (I) is disseminated using an air-freshener device.

8. A pharmaceutical composition prepared using a compound of formula (I) as defined in claim 1.

9. A compound of formula (I) 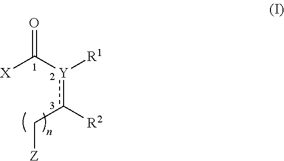
wherein n is 0 or 1;
R¹ is linear or branched C₃-C₇ alkyl, benzyl or pyridylmethyl;
R² is hydrogen, C₁-C₄ alkyl, or C₂-C₄ alkenyl; or
R² forms together with the carbon atom to which it is attached a carbonyl group;
X is selected from hydrogen, C₁-C₃ alkyl, C₁-C₃ alkoxy, or NR³R⁴ wherein R³ and R⁴ independently are selected from hydrogen, or C₁-C₃ alkyl; with the proviso that if R¹ is pyridylmethyl then X is not methyl;
I) Z is a 3-6 membered monocyclic or 6-10 membered bicyclic hydrocarbon ring wherein one or two C atom(s) are replaced by a hetero atom selected from S, O, or N;
with the proviso that for Z=pyridyl, R¹ is not benzyl;
II) Z is a 3-6 membered monocyclic or 6-10 membered bicyclic hydrocarbon ring wherein one or two C atom(s) are replaced by a hetero atom selected from S, O, or N, and the ring is substituted with up to 5 groups selected from hydroxyl, CN, halogen, mono-, di-, or trihalogenomethyl, C₁-C₃ alkoxy, C₁-C₃ alkyl, —COOR, or —OCOR wherein R is hydrogen, methyl, ethyl, propyl or isopropyl;
III) Z is C₁-C₄ alkoxy, with the proviso that for Z=ethoxy, R¹ is not benzyl; or
IV) Z is cyclopropyl;
and Y represents a N- or C-atom with the proviso that for X═NR³R⁴, Y═C and the dotted line represents together with the carbon-carbon bond a double bond, either in E or Z configuration, or a single bond.

10. A compound according to claim 9 selected from the list consisting of N-pentyl-N-(pyridin-3-ylmethyl)acetamide; methyl pentyl(pyridin-3-ylmethyl) carbamate; N-pentyl-N-(pyridin-4-ylmethyl)acetamide; methyl pentyl(pyridin-4-ylmethyl)carbamate; N-(cyclopropylmethyl)-N-pentylacetamide; methyl cyclopropylmethyl(pentyl)carbamate; methyl bis(pyridin-3-ylmethyl) carbamate; methyl bis(pyridin-2-ylmethyl)carbamate; N-pentyl-N-(2-(pyridin-2-yl)ethyl)acetamide; methyl pentyl(2-(pyridin-2-yl)ethyl)carbamate; N-pentyl-N-(2-(pyridin-3-yl)ethyl)acetamide; methyl pentyl(2-(pyridin-3-yl)ethyl)carbamate; N-pentyl-N-(2-(pyridin-4-yl)ethyl)acetamide; methyl pentyl(2-(pyridin-4-yl)ethyl)carbamate; N-pentyl-N-(pyridin-2-ylmethyl)acetamide; methyl pentyl(pyridin-2-ylmethyl) carbamate; N-(2-(1H-imidazol-4-yl)ethyl)-N-pentylacetamide; N-acetyl-N-pentylcyclopropane carboxamide; tert-butyl acetyl(pentyl)carbamate; N-(cyclopropylmethyl)-N-pentylformamide; (E)-2-benzylidene-N-methylheptanamide; (E)-2-(cyclopropylmethylene)-N,N-dimethylheptanamide; and (E)-2-(cyclopropylmethylene)heptanamide.

11. A tobacco product comprising a compound of formula (I) as defined in claim 4.

12. A method comprising the step of dissemination a compound of formula (I) as defined in claim 4 into a room in the presence of tobacco smoke.

13. A method according to claim 12 wherein the compound of formula (I) is disseminated using an air-freshener device.

14. A pharmaceutical composition prepared using a compound of formula (I) as defined in claim 4.
